(19) United States
(12) Patent Application Publication
(10) Pub. No.: US 2016/0349238 A1
(43) Pub. Date: Dec. 1, 2016

(54) METHOD FOR MEASURING HUMAN EXHALED AIR

(71) Applicant: IMSPEX DIAGNOSTICS LTD, Abercynon RCT (GB)

(72) Inventors: Stefanie SIELEMANN, Dortmund (DE); Thomas WORTELMANN, Dortmund (DE)

(73) Assignee: IMSPEX DIAGNOSTICS LTD, Abercynon RCT (GB)

(21) Appl. No.: 15/038,086

(22) PCT Filed: Nov. 21, 2014
(86) PCT No.: PCT/EP2014/075236

§ 371 Date: May 20, 2016

(30) Foreign Application Priority Data
Nov. 22, 2013 (DE) 10 2013 112 921.8

(51) Int. Cl. G01N 33/497; G01N 27/62; G01N 30/72

(52) U.S. Cl. CPC G01N 33/497; G01N 30/72; G01N 27/622

(57) ABSTRACT

A method for measuring human exhaled air by means of gas chromatography and ion mobility spectrometry, wherein an exhaled air sample enters a sample loop via a sample inlet and a multi-port valve and is subsequently conveyed by means of a carrier gas from the sample loop, via the multi-port valve through a gas chromatographic column, into an ion mobility spectrometer, and measured, which method is to provide reliable and accurate measurement results. This object is achieved in that the following steps are carried out before an exhaled air sample is introduced into the sample loop: (a) first flushing at least the gas chromatographic column, the ion mobility spectrometer and the sample loop with a flushing gas and then switching the multi-port valve in such a way that the flushing gas enters the ion mobility spectrometer and is measured; (b) then stopping the supply of flushing gas and switching the multi-port valve in such a way that ambient air flows through the gas chromatographic column into the ion mobility spectrometer and is measured; (c) then flushing at least the gas chromatographic column, the ion mobility spectrometer and the sample loop with humidified flushing gas and switching the multi-port valve in such a way that the humidified flushing gas enters the ion mobility spectrometer and is measured; and (d) subsequently stopping the supply of humidified flushing gas, conducting an exhaled air sample into the sample loop, conveying the sample, by means of the carrier gas, through the gas chromatographic column into the ion mobility spectrometer, and measuring the sample.

The invention relates to a method for measurement of human exhaled air by means of gas chromatography/ion-mobility spectrometry; in which an exhaled-air sample is passed via a sample inlet and a multi-way valve into a sample loop and subsequently is conveyed by means of a carrier gas out of the sample loop via the multi-way valve through a gas-chromatographic column into an ion-mobility spectrometer and measured.

The detection of volatile organic compounds in human breath by means of different analytical methods has been frequently described. Even ion-mobility spectrometers as well as their combinations with gas-chromatographic pre-separation have already been used in research projects.

For example, a method with the features of the preamble of claim 1 is known from Journal of Chromatography A, 1084 (2005), pages 145 to 151.

For routine use in hospitals, healthcare institutes as well as medical practices, however, these analytical methods are still suitable to only a limited extent, since a reliable measurement is not assured and frequently measurement of the background (cleaning agents, other impurities) or a mistaken assignment of the measured results takes place. Various volatile compounds (e.g. ketones) and other components also elute from the gas-chromatographic column because of their adsorption on the lines of the measuring system, frequently only in subsequent measurements, and falsify the actual results.

To this extent, the results of this otherwise promising method cannot yet meet the requirements of routine use, and so gas chromatography/ion-mobility spectrometry, the advantages of which lie in its sensitivity, has problems with respect to its scientific acceptance.

The task of the invention is to specify a method for measurement of human exhaled air by means of gas chromatography/ion-mobility spectrometry, which delivers reliable and correct measured results.

This task is accomplished according to the invention with a method of the type described in the introduction, by the fact that, before the feed of an exhaled-air sample into the sample loop

a) firstly at least the gas-chromatographic column, the ion-mobility spectrometer and the sample loop are swept with a purge gas and the multi-way valve is then switched such that the purge gas is passed into the ion-mobility spectrometer and measured,

b) thereafter the purge-gas feed is ended and the multi-way valve is switched such that ambient air is passed through the gas-chromatographic column into the ion-mobility spectrometer and measured,

c) then at least the gas-chromatographic column, the ion-mobility spectrometer and the sample loop are swept with humidified purge gas and the multi-way valve is switched such that the humidified purge gas is passed into the ion-mobility spectrometer and measured,

d) the feed of the humidified purge gas is ended and an exhaled-air sample is injected into the sample loop and conveyed by means of the carrier gas through the gas-chromatographic column into the ion-mobility spectrometer and measured.

The method according to the invention is therefore designed in such a way that contaminations before an actual measurement and erroneous measurements are prevented by the fact that, before the actual breath measurement, firstly the previously described steps are carried out in succession, i.e. in a first method step a clearance measurement of the sensors (gas-chromatographic column and ion-mobility spectrometer) without sample measurement, in a second step the ambient air and in a third step a clearance measurement of the system with humidified purge gas as sample are performed, so that, after removal of contaminations by the purge process, the measuring-system parameters as well as the ambient conditions are taken into consideration, i.e. both the measuring-system parameters (e.g. reaction-ion peak (RIP)) and also the composition or condition of the ambient air and the moisture content are recorded, so that these recorded parameters are taken into consideration in the subsequent measurement and evaluation of the exhaled-air sample.

In order to avoid breath-sample contamination after completion of the measurement, it is preferably provided that, after measurement of a breath sample, at least the gas-chromatographic column, the ion-mobility spectrometer, the sample loop and the sample inlet are swept with a purge gas. This sweeping is maintained sufficiently long until a new breath-measurement process is scheduled or the measuring device is turned off.

In a particularly preferred embodiment, it is provided that a spirometer is used as the sample inlet. A medical spirometer, the sensors of which are integrated in the hand-held housing for precise and direct recording of CO2/O2 and volume flow, is preferably used for validatable and reproducible sampling. The flexible transition tubes to the actual measuring device are flushed with purge gas and preferably heated outside a measuring process, in order to prevent condensation and to be able to clean contamination.

In this connection it is preferably provided that the flow rate of the exhaled air is monitored by the spirometer and, in case of a drop below a predetermined limit value, the exhaled-air feed into the sample loop is interrupted. For example, if a patient is unable to blow sufficient exhaled air into the spirometer, the exhaled-air feed is interrupted in order to prevent ambient air from passing into the measuring system. As the limit value for predetermined flow conditions of the spirometer, it is possible, for example, to set a time interval. Thus it is possible to provide, for example, that it is necessary to exhale continuously into the mouthpiece of the spirometer for only several seconds. If the exhalation process is interrupted or prematurely ended, the exhaled-air feed is interrupted.

Alternatively, it may be provided that the flow rate of the exhaled air is monitored by the spirometer and the exhaled-air feed into the sample loop is released only after exceedance of a predetermined limit value. A patient is then able to exhale several times in smaller volumes, which are then added, so that a sufficiently large sample volume (breath), which is passed into the sample loop, is available.

Furthermore, it is preferably provided that a calibration or test gas or an external breath sample is fed from a sampling vessel via an additional gas inlet to the multi-way valve.

The invention will be explained in more detail in the following by way of examples on the basis of the drawing. This shows, in

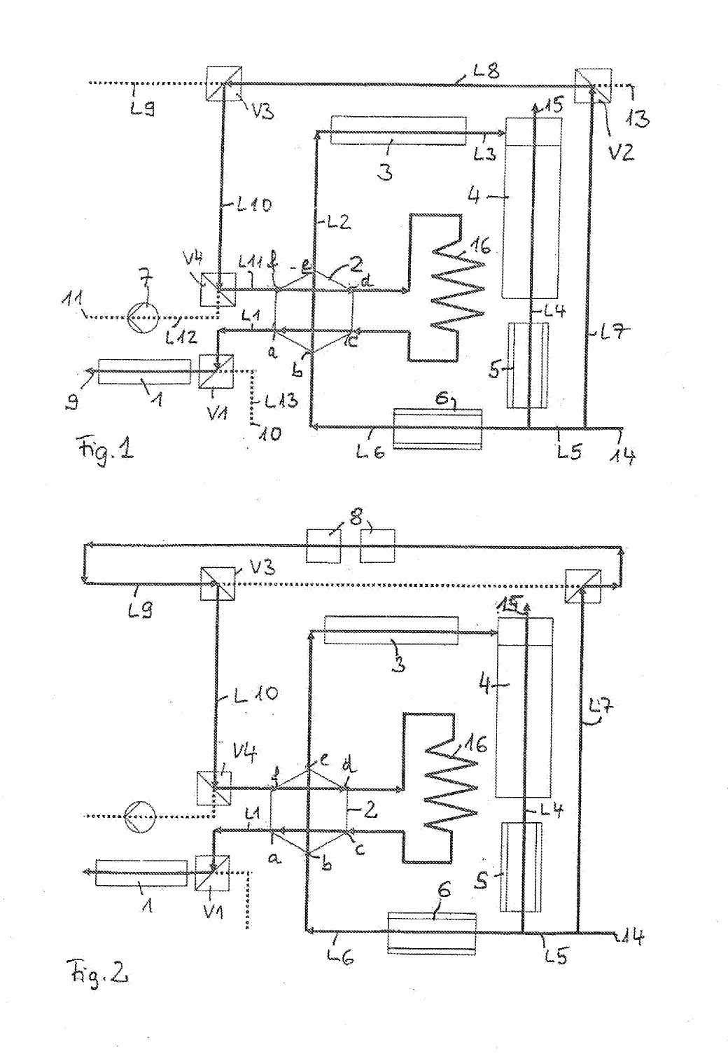

FIGS. 1 to 6 a measuring device, in schematic representation, for measurement of human exhaled air by means of gas chromatography/ion-mobility spectrometry in various measurement-sequence stages.

The measuring device is provided firstly with a spirometer 1, which is in communication via a changeover valve V1 and a line L1 with a multi-way valve, in the exemplary embodiment a 6-way valve 2. The six inputs or outputs of the 6-way valve 2 are denoted a, b, c, d, e and f. A sample loop 16 is connected to the input or output c, d of the 6-way valve 2. The output e of the 6-way valve 2 is in fluid communication via a line L2 with a gas-chromatographic column 3, preferably a multi-capillary column, the output of which is in communication via a line L3 with the ionization chamber of an ion-mobility spectrometer 4. A line L4, which is equipped with an electronic pressure regulator 5 for the drift gas, is connected to the drift-gas input of the ion-mobility spectrometer 4. The line L4, as a branch line, is in fluid communication with a gas-feed line L5, which at the end is in communication with a gas inlet 14. Furthermore, a line L6, which is equipped with an electronic pressure regulator 6 for a carrier gas, is branched off from line L5. Line L6 ends at the inlet b of the 6-way valve 2.

In the exemplary embodiment, therefore, only one gas inlet 14 is provided for the carrier gas and the drift gas, i.e. these are identical in the exemplary embodiment, e.g. nitrogen or synthetic air. Furthermore, a line L7, which can be in communication via a changeover valve V2 with a gas outlet 13 or a line L8, branches off from line L5. Line L8 is in communication via a further changeover valve V3 with a line L9 or a line L10. The line L10 is in communication via a changeover valve V4 either with a line L11, which is connected to the port f of the 6-way valve 2, or with a line L12, in which a pump 7 is disposed and which ends in a sample outlet 11. The line L9 is, as follows from FIG. 2, connected to external water bottles 8 and discharges at changeover valve V2.

A sample input at the spirometer 1 is denoted by 9; a calibration input 10 is connected via a line L13 to the changeover valve V1 of the spirometer 1. Furthermore, the gas outlet of the ion-mobility spectrometer 4 is denoted by 15.

Various method sequence stages are illustrated in FIGS. 1 to 6. Depending on valve position of the 6-way valve 2 and of the further valves V1, V2, V3, V4, respectively only individual lines are released; the released, i.e. active lines are illustrated in FIGS. 1 to 6 as solid lines. In contrast, inactive lines are illustrated as dotted lines.

The drift gas for the purging and achievement of optimum results of the ion-mobility spectrometer 4, feedable via the gas inlet 14, is controlled by the electronic pressure regulator 5. The sample carrier gas, which is injected via the gas-chromatographic column 3 and then into the ion mobility spectrometer 4, is controlled by the electronic pressure regulator 6. Both gases (drift and sample carrier gas), i.e. nitrogen or synthetic air, are guided on separate paths to the gas outlet 15. Both the ion-mobility spectrometer 4 and the gas-chromatographic column 3 as well as the 6-way valve 2 are preferably temperature-controlled.

As long as no measurement of the breath or else a test/calibration gas is taking place, the measuring system is being swept with purge gas. For cleanliness of the overall system, the purge gas additionally sweeps through the spirometer 1 as well, in order to prevent adsorptions of substances of previous measurements on the internal lines L1, L7, L8, L10 and L11, the valves V1 and V4, the sample loop 16 and the ports a, b, c, d, e, f of the 6-way valve 2.

A gas sample is sucked into the system by means of the pump 7. The sampling of the breath can take place directly by exhalation into a replaceable mouthpiece inserted into a holder of the spirometer 1. The sample is transported to the 6-way valve 2 via the preferably heated line L1. Alternatively, and for calibration purposes, the sample may also be introduced from a gas bottle or a gas-sample container via the calibration input 10 into the line L13.

For measurement of a gas sample from the breath or a test-gas source, carrier gas sweeps continuously through the gas-chromatographic column 3 in the basic setting of the 6-way valve 2, which is illustrated in FIG. 1. By means of the pump 7, a sample gas is sucked via the spirometer 9 or via the calibration input 10 through the sample loop 16. In this position, the sample gas is guided from the spirometer 1 or calibration inlet 10 directly to the gas outlet 11.

For performance of the actual measurement, the sample is transported in the sample loop 16 to the gas-chromatographic column 3 and subsequently to the ion-mobility spectrometer 4 by switching of the 6-way valve 2. In this way the carrier gas conveys the breath sample into the sample loop 16 and further to the gas-chromatographic column 3, where the substances present in the sample are separated according to their retention time. The elating substances are injected via the line L3 into the ionization chamber of the ion-mobility spectrometer 4.

A medical spirometer 1, the sensors of which are integrated in the hand-held housing for more precise and direct recording of CO2/O2 and volume flow, is used for validatable and reproducible sampling. The connection line L1 is flushed with purge gas in the basic position and is heated, in order to prevent condensation and to be able to clean contaminations. By virtue of communication between the spirometer 1 and the controller of the measuring device, the timing of a switching of the 6-way valve 2 and thus of the sampling can be varied/optimized, depending on the analytical problem, by means of CO2/O2 or volume-flow measurement of the breath and saved in the program sequence.

The various system settings of the measuring system and thus of the measuring method sequence are as follows:

The basic setting is illustrated in FIG. 1, wherein purge gas (drift as well as sample carrier gas) flows from the gas inlet 14 via the active lines (solid lines) on the one hand as drift gas through the ion-mobility spectrometer 4, on the other hand via the correspondingly switched inputs and outputs b, e of the 6-way valve 2 as sample gas through the gas-chromatographic column 3 and the ion-mobility spectrometer 4 and further via the ports f, d, c and a of the 6-way valve 2 connected to one another through the sample loop 16 and the spirometer 1. In this basic position, therefore, purge gas is flowing through all system components.

At the beginning of a breath measurement, a clearance measurement of the system is performed in this basic position according to FIG. 1, i.e. during the sweeping of the system components, the purge gas is passed as sample gas, so to speak, into the ionization chamber of the ion-mobility spectrometer 4 and the purge gas is measured in the ion-mobility spectrometer 4.

The measured values are saved accordingly in the system controller and are taken into consideration in the later sample measurement or measurement evaluation.

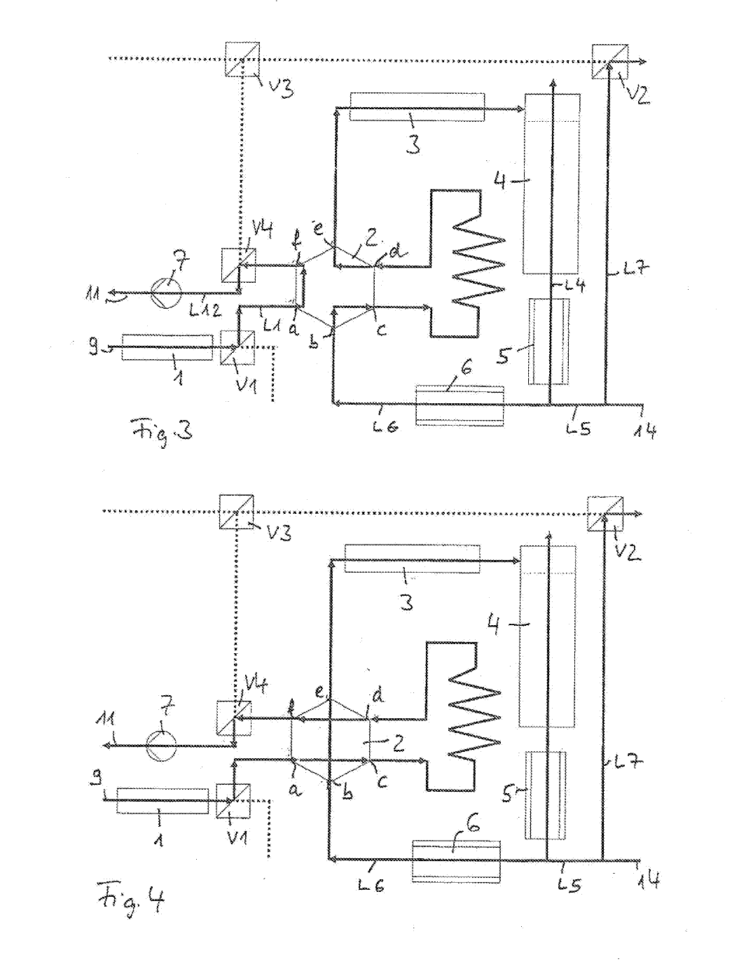

In a second method step, the purge-gas feed is ended and the multi-way valve 2 is switched such that ambient air is passed through the gas-chromatographic column 3 into the ion-mobility spectrometer 4 and measured there. For this purpose, the pump 7 sucks ambient air through the spirometer 1 to the gas-tight sample loop 16. The multi-way valve 2 is then in the switched position illustrated in FIG. 4. Then the 6-way valve is switched into the position illustrated in FIG. 3, so that a sample, in this case ambient air, is transported to the gas-chromatographic column 3 and further to the ion-mobility spectrometer and the measurement data are recorded. The measured data of the ambient air are correspondingly processed.

In a third method step, then at least the gas-chromatographic column 3 of the ion-mobility spectrometer 4 and the sample loop 16 are purged with humidified purge gas. This situation is illustrated in FIG. 2; the purge gas entering via gas inlet 14 is guided via the line L8 through external water bottles 8 and humidified, and thus is also passed into the sample loop 16. Then the 6-way valve 2 is switched into the position illustrated in FIG. 3, so that the sample, in this case humidified N2 or synthetic air, for example, is transported further to the gas-chromatographic column 3 and to the ion-mobility spectrometer 4 and the measurement data are recorded. The measurement data of this third process step are also saved and taken into consideration correspondingly during the later evaluation of the breath sample.

Then the purge-gas feed is ended and in the last step an exhaled-air sample of a patient is injected into the sample loop 16. In the switched position of the 6-way valve 2 illustrated in FIG. 4, the breath sample is passed from the spirometer 1 into the sample loop 16. For this purpose, the breath sample is sucked in by the pump 7.

In the process, the patient is prompted by software techniques to breath continuously into the mouthpiece of the spirometer 1, in order to fill the sample loop 16. For example, it is necessary to breath continuously for 6 seconds into the mouthpiece of the spirometer 1. If the patient is unable to follow the specified exhalation procedure and/or if this is interrupted during the specified time interval, the valve V1 is reset and the pump controller of the pump 7 interrupts the suction process. This prevents ambient air from passing into the system.

In contrast, if the exhaled air is passed correctly into the sample loop 16, after filling of the same the multi-way valve 2 is switched into the position according to FIG. 3 and the breath sample is conveyed by the carrier gas through the gas-chromatographic column 3 into the ion-mobility spectrometer 4 and measured there. Then the measured-value evaluation of the breath sample takes place with consideration of the previous measurements.

If a patient is unable to breath continuously into the mouthpiece of the spirometer 1 for six seconds, for example, the possibility exists through software techniques of adding the individual smaller volumes, in that the valve V1 is reset after each exhalation procedure and only when a sufficiently large total volume is available is the 6-way valve 2 switched and the sample passed from the sample loop 16 into the column 3 and subsequently into the ion-mobility spectrometer 4.

After completion of the feed of the breath sample, a changeover to the basic position according to FIG. 1 takes place, i.e. purge gas flows through the system components, before a new measurement cycle begins or the measuring device is turned off.

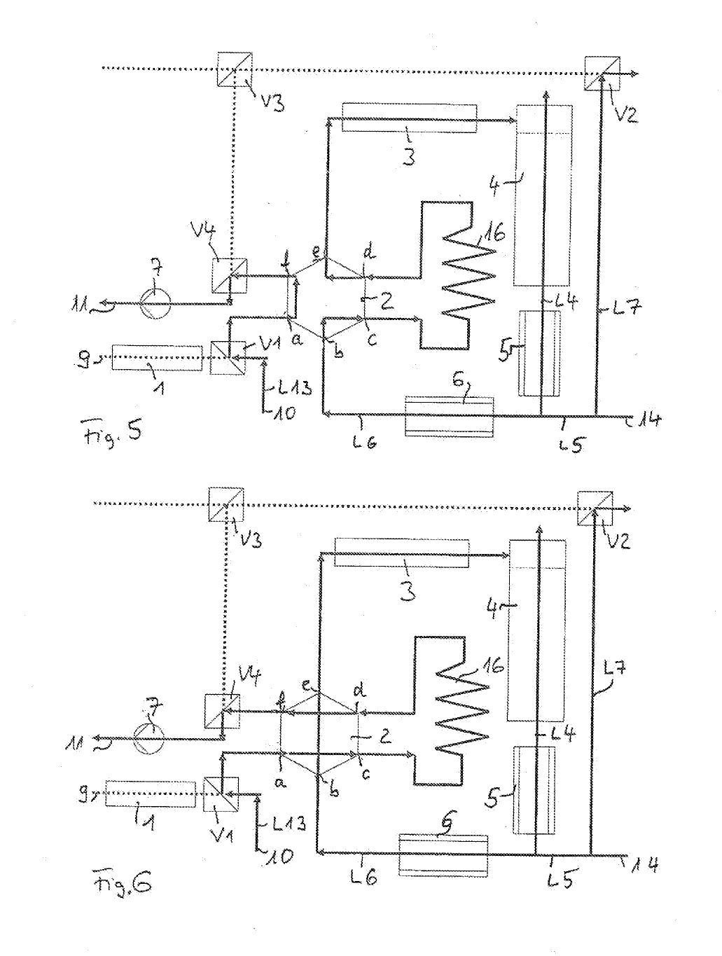

FIGS. 5 and 6 show an additional method embodiment, which is used for measurement of test/calibration gas or samples from external sample containers with feed via the calibration input 10. Starting from the basic position according to FIG. 1, the purge-gas feed is ended and the valve V1 between the spirometer 1 and the calibration input 10 is switched from the spirometer 1 to the calibration input 10 and the gas flow is changed over. In this case the multi-way valve 2 is initially in the position according to FIG. 6. Thus the pump 7 sucks from the calibration input 10 into the sample loop 16. Then the 6-way valve is switched into the position illustrated in FIG. 5, so that the sample, in this case test or calibration gas or sample gas, is transported from an external container to the gas-chromatographic column 5 and further to the ion-mobility spectrometer 4 and the measured data are recorded. After completion of this additional method step, the measuring system is reset again to its basic position (purge mode) according to FIG. 1.

1. Method for measurement of human exhaled air by means of gas chromatography/ion-mobility spectrometry, in which an exhaled-air sample is passed via a sample inlet and a multi-way valve into a sample loop and subsequently is conveyed by means of a carrier gas out of the sample loop via the multi-way valve through a gas-chromatographic column into an ion-mobility spectrometer and measured,
wherein,
before the feed of an exhaled-air sample into the sample loop
a) firstly at least the gas-chromatographic column, the ion-mobility spectrometer and the sample loop are swept with a purge gas and the multi-way valve is then switched such that the purge gas is passed into the ion-mobility spectrometer and measured,
b) thereafter the purge-gas feed is ended and the multi-way valve is switched such that ambient air is passed through the gas-chromatographic column into the ion-mobility spectrometer and measured,
c) then at least the gas-chromatographic column, the ion-mobility spectrometer and the sample loop are swept with humidified purge gas and the multi-way valve is switched such that the humidified purge gas is passed into the ion-mobility spectrometer and measured,
d) the feed of the humidified purge gas is ended and an exhaled-air sample is injected into the sample loop and conveyed by means of the carrier gas through the gas-chromatographic column into the ion-mobility spectrometer and measured.

2. Method according to claim 1,
wherein,
after measurement of a breath sample, at least the gas-chromatographic column, the ion-mobility spectrometer, the sample loop and the sample inlet are swept with a purge gas.

3. Method according to claim 1,
wherein
a spirometer is used as sample inlet.

4. Method according to claim 3,
wherein
the flow rate of the exhaled air is monitored by the spirometer and, in case of a drop below a predetermined limit value, the exhaled-air feed into the sample loop is interrupted.

5. Method according to claim 3,
wherein
the flow rate of the exhaled air is monitored by the spirometer and the exhaled-air feed into the sample loop is released only after exceedance of a predetermined limit value.

6. Method according to claim 1,
wherein
a calibration or test gas or an external breath sample is fed from a sampling vessel via an additional gas inlet to the multi-way valve.
